(19) United States
(12) Patent Application Publication
(10) Pub. No.: US 2022/0023183 A1
(43) Pub. Date: Jan. 27, 2022

(54) COMPOSITION COMPRISING A POLYSACCHARIDE, A POLYOL AND A SPECIFIC ESTER

(71) Applicant: L'OREAL, Paris (FR)

(72) Inventors: Emmanuelle PORTOIS, Chevilly La Rue (FR); Julien LABOUREAU, Chevilly La Rue (FR); Pamella WANG, Chevilly La Rue (FR)

(21) Appl. No.: 17/413,091

(22) PCT Filed: Dec. 20, 2019
(86) PCT No.: PCT/EP2019/086737

§ 371 Date: Jun. 11, 2021

(30) Foreign Application Priority Data
Dec. 20, 2018 (FR) 1873644

(51) Int. Cl. A61K 8/73; A61K 8/34; A61K 8/37; A61Q 19/10

(52) U.S. Cl. CPC A61K 8/73; A61Q 19/10; A61K 8/375; A61K 8/345

(57) ABSTRACT

The present invention relates to a composition, particularly a cosmetic composition, comprising, in a physiologically acceptable medium: at least one polysaccharide comprising rhamnose, at least one polyol, and at least 1% by weight relative to the total weight of composition, at least one ester of fatty acid and of polyglycerol comprising from 5 to 9 glycerol patterns.

This invention relates to a composition, preferably cosmetic, comprising a polysaccharide comprising rhamnose, a polyol and a specific ester.

The skin is a tissue in which cells are contiguous and firmly attached to each other. Skin tissue forms an external coating comprising sebaceous or sudoriferous glands, and hair follicles. The skin, and particularly the scalp, are continuously renewed epithelia. Renewal, or desquamation, is a coordinated and finely regulated process leading to the elimination of surface cells, insensibly and invisibly.

The human skin is composed of two compartments, namely a surface compartment (the epidermis) and a deep compartment (the dermis).

The epidermis is conventionally divided into a base layer of keratinocytes making up the germinative layer of the epidermis, a so-called spiny layer composed of several layers of polyhedral cells located on the germinative layers, one to three layers called granular layers composed of flat cells containing distinct cytoplasmic inclusions, keratohyalin grains and finally a set of upper layers called horny layers (stratum corneum), composed of keratinocytes called corneocytes at the terminal stage of their differentiation.

Corneocytes are anucleate cells composed principally of a fibrous material containing cytokeratins, surrounded by a corneal envelope. There is permanent production of new keratinocytes to compensate for the continuous loss of epidermal cells in the stratum corneum according to a mechanism called desquamation.

However, an unbalance between the production of cells in the base layer and the desquamation rate can lead to the formation of scales on the skin surface. Similarly, for various reasons, a deficit of terminal differentiation of cells in the stratum corneum can lead to the formation of large, thick clumps of cells, visible to the naked eye and called “dander”, or in other situations, to thinning of the stratum corneum. This can cause fragility of the barrier properties of the epidermis, chronic dehydration of the stratum corneum, loss of mechanical elasticity, tightness, and make the skin lose its luster and transparency. Among examples of factors conducive to this alteration of the surface quality of the skin, mention may be made of stress, the winter period, excess sebum, a hydration disorder; this can also be the case for dry skin of elderly subjects.

Thus, fragility of the skin barrier can occur in the presence of external aggression such as irritants (detergents, acids, bases, oxidants, reducing agents, concentrated solvents, noxious gases or fumes), mechanical actions (friction, shocks, abrasion, surface tearing, projection of dust, particles, shaving or epilation), thermal or climatic unbalances (cold, dryness, radiation), xenobiotic unbalances (undesirable micro-organisms, allergens) or internal aggressions of the psychological stress type.

One of the critical steps in the terminal differentiation process of the stratum corneum is cross-linking of proteic precursors of the cornified envelope. This phenomenon plays an essential role in the development and maintenance of skin cohesion and physical properties of the skin such as the barrier function.

The cornified envelope is an essential component of corneocytes.

Maturing of the cornified envelope from the deep layers to surface layers of the stratum corneum can be characterized by morphological and biophysical or mechanical parameters.

Hydrating agents conventionally used such as moisturizers, hydrating polymers or fatty bodies such as petroleum jelly, temporarily modify the surface properties of the skin. These active agents can increase the mechanical suppleness of the stratum corneum, increase its state of hydration and/or improve the microrelief of the skin by the formation of a surface film on the skin. In general, these effects are not remanent in time and only last for a few hours. Furthermore, after the skin has been cleaned, these active agents are eliminated and the effect of increased mechanical suppleness of the skin, improved skin texture or optical properties of the skin disappear.

Furthermore, the use of-film forming agents on the skin, and particularly the use of moisturizing polysaccharides such as carrageenan, often leads to a skin “tightening” effect, an increase in the elastic modulus of the skin; this increased surface stiffness causes discomfort of the skin.

Therefore there is a need for active agents that improve the state of hydration of the skin, particularly dry skin or old skin, avoiding tightness and the feeling of discomfort during application on the skin.

There is also a need for compositions that confer a plumping effect and/or a bouncy appearance on the skin. “Bouncy appearance” means an effect of remodelling the skin. The skin is smoother and has a more fleshy appearance, that remains even after pressing on the skin with a finger.

The inventors have now discovered that the association of a particular polysaccharide, i.e. comprising rhamnose, with a polyol and a specific ester, can satisfactorily increase the suppleness of the skin and confer a plumping effect and a bouncy appearance on it.

Thus, the purpose of the present invention is a composition comprising, in a physiologically acceptable medium:

at least one polysaccharide comprising rhamnose,

at least one polyol, and

at least 1% by weight relative to the total weight of the composition, at least one ester of fatty acid and of polyglycerol comprising from 5 to 9 glycerol patterns.

The composition according to the invention is preferably cosmetic.

“Physiologically acceptable” means a medium compatible with keratin materials.

Another purpose of this invention is a method of cosmetic treatment of keratin fibers, preferably the skin, comprising application of a composition according to the invention on said keratin fibers.

Another purpose of this invention is cosmetic use of a composition according to the invention to make the skin more supple, particularly the stratum corneum.

Another purpose of the invention is a composition preferably cosmetic, comprising, in a physiologically acceptable medium:

at least one polysaccharide comprising rhamnose, and

at least at least one hydrophilic active agent, preferably chosen from the C-glycoside

derivatives of general formula (F) below:

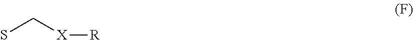

in which:

R denotes an unsubstituted linear C1-C4 alkyl radical, especially C1-C2, in particular methyl;

S represents a monosaccharide chosen from D-glucose, D-xylose, N-acetyl-D-glucosamine or L-fucose, and in particular D-xylose;
X represents a group chosen from —CO—, —CH(OH)—, —CH(NH2)-, and preferentially a —CH(OH)— group;

as well as their cosmetically acceptable salts, their solvates such as hydrates and their optical isomers; and preferably chosen from C-beta-D-xylopyranoside-2-hydroxy-propane and C-alpha-D-xylopyranoside-2-hydroxy-propane.

Polysaccharide Comprising Rhamnose

The composition according to the invention comprises at least one polysaccharide comprising rhamnose.

Preferably, the polysaccharide according to the invention comprises rhamnose varying from 10% to 100% by weight relative to the total weight of polysaccharide, preferably from 20% to 70% by weight, and more preferably from 40% to 60% by weight.

Preferably, the polysaccharide according to the invention is not sulfated. “Not sulfated” means that the sulfation ratio of the polysaccharide is less than 0.5% by weight, preferably less than 0.1% by weight relative to the weight of polysaccharide. Preferably, the sulfation ratio is zero.

Thus preferably, the polysaccharide according to the invention is such that the repetitive elements from which it is made predominantly contain rhamnose. Preferably, the repetitive elements comprise at least components with general formula I:

(Rh;Rh;Rh*;U;O)ₙ (I)

wherein Rh is a rhamnose molecule, Rh* is a rhamnose molecule fixed in a branched manner, O is a molecule of a hexosidic or pentosidic sugar, U is a molecule of uronic acid and n is between 1 and 100, and preferably between 5 and 65.

“Repetitive elements predominantly containing rhamnose” means a branched chain comprising at least 50% of rhamnose in the D and/or L series, and its a and/or β isomers.

The sugar O may in particular be chosen from among fucose, galactose, ribose, arabinose, xylose and mannose.

Uronic acid U means any hexose oxidized on its primary alcohol function into carboxylic acid, and particularly glucuronic acid, galacturonic acid, mannuronic acid or iduronic acid.

According to one particular embodiment of the invention, the branched rhamnose molecule can be fixed by an osidic bond from its carbon 1 on a free carbon of one among the sugar molecule O or uronic acid molecule U or rhamnose molecule Rh of the saccharidic chain, particularly carbons 2 or 3.

According to another particular embodiment, the repetitive elements can be composed in particular by the sequence with general formula II:

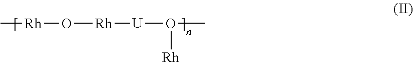

wherein Rh is a rhamnose molecule, O is a hexosidic or pentosidic sugar molecule, U is a uronic acid molecule and the rhamnose branch onto the ose O consists of an osidic bond (1→2) or (1→3).

According to one particularly interesting embodiment, the sugar 0 is galactose and the uronic acid U is glucuronic acid. Preferably, the sequence has a chain containing 3 rhamnose molecules, one of which is branched, 2 galactose molecules and one glucuronic acid molecule. According to formula II, n represents a value such that this polysaccharide has a molecular weight of the order of 50,000 daltons. It can be obtained from Klebsiella type bacteria cultures, particularly Klebsiella pneumoniae and particularly the I-714 strain (deposed at CNCM—Collection Nationale de Culture de Microorganismes (National Microorganisms Culture Collection)—as number I-714) according to a method described below. Advantageously, this polysaccharide has the rhamnose branch on galactose in position V. It is found that this polysaccharide is composed particularly of the following repetitive unit: →4)-α-L-Rhap(1→3)-β-D-Galp(1→2)-α-L-Rhap(1→4)-β-D-GlcpA(1→3)-[α-L-Rhap(1→2)]-α-D-Galp(1→.

Hydrolysis of this polysaccharide also makes it possible to obtain a mixture of fractions with lower molecular weight, particularly majority fractions of 5,000 daltons and 13,000 daltons, possibly purifiable and particularly interesting according to the invention.

According to another particular embodiment, the repetitive elements can be composed in particular by the sequence with general formula III:

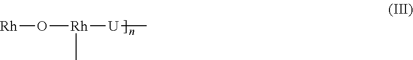

wherein Rh is a rhamnose molecule, O is a hexosidic or pentosidic sugar molecule, U is a uronic acid molecule and rhamnose is branched onto the rhamnose by an osidic bond (1→3). According to one particularly interesting embodiment, the sugar O is glucose and the uronic acid U is glucuronic acid, preferably a chain containing 3 rhamnose molecules including one branched molecule, one glucose molecule and one glucuronic acid molecule. Such a polysaccharide can be obtained in particular according to the method described below from a culture of Klebsiella planticola type bacteria, particularly the I-2743 strain (deposited at CNCM as number 1-2743). Advantageously, such a polysaccharide has the rhamnose branch on the rhamnose in position III. It is found that this polysaccharide is composed more particularly of the following repetitive unit: →3)-β-L-Rhap(1→4)-β-D-Glcp(1→2)-[α-L-Rhap(1→3)]-α-L-Rhap(1→4)-α-D-GlcpA(1→.

Hydrolysis of this polysaccharide can also result in a mixture of fractions with lower molecular weight, particularly the majority fraction of 5,000 daltons, possibly purifiable and particularly interesting according to the invention.

In general, the polysaccharides according to the invention can be of bacterial or vegetable origin. They can be obtained by classical polysaccharide production techniques (chemical synthesis, enzymatic extraction from exopolysaccharides). According to one advantageous embodiment, the polysaccharides are exopolysaccharides obtained by fermentation of a bacterial strain producing them, of the encapsulated bacteria type, according to a production method like that described in detail in patent FR264522. This method is defined in that a Klebsiella type bacteria strain is put into culture in a nutrient medium comprising a carbon source, a preferential nitrogen source and appropriate mineral salts, at a pH of about 6 to 8, at a temperature of about 30 to 35° C., while stirring and under aeration, for 4 to 12 days. The carbon/nitrogen ratio is advantageously more than 5 so as to favor secretion of the polysaccharide. The polysaccharide can then be isolated by submitting the fermentation medium to heat treatment at about 70-120° C. for about 10 minutes to 1 hour, then by separating it, for example by centrifuging it cold. The exopolysaccharides and cellular polysaccharides are all contained in the clear float phase. If necessary, the polysaccharides can be purified by precipitation by the addition of a non-solvent organic liquid such as acetone or a lower alcohol such as ethanol or propanol, and separated by filtration or centrifuging before being dried.

The isolated polysaccharides can thus be easily incorporated into a composition, as is or in hydrolyzed form. In this case, the hydrolysis can be done before drying using known methods such as acid hydrolysis. It can be done using a frequently used proton donor such as hydrochloric acid, at a temperature varying between 50 and 100° C. for between 30 minutes and 4 hours, depending on the required size of the fractions. The oligosaccharidic fractions thus obtained can be recovered and purified if necessary, using classical methods.

This protocol can be done using bacterial strains producing exopolysaccharides rich in rhamnose, and particularly encapsulated bacteria. According to one preferred embodiment of the invention, a strain of Klebsiella bacteria will be used, preferably Klebsiella pneumoniae or Klebsiella planticola.

Preferably, the repetitive unit of the polysaccharide (exopolysaccharide) according to the invention is that produced by Klebsiella pneumoniae 1-714 and called Rhamnosoft®:

The composition of Rhamnosoft® corresponds to a polymer with a branched structure, with molecular weight of the order of 50,000 daltons, and with a saccharidic sequence comprising three rhamnose molecules (I, III, VI), two galactose molecules (II, V) and one glucuronic acid molecule (IV). Therefore rhamnose makes up 50% of the polysaccharide. The polysaccharide has a rhamnose VI branch on the galactose in position V.

In this case the structure of the repetition unit is:

→4)-α-L-Rhap(1→3)-β-D-Galp(1→2)-α-L-Rhap(1→4)-β-D-GlcpA(1→3)-[α-L-Rhap(1→2)]-α-D-Galp(1→.

It corresponds to the following detailed formula:

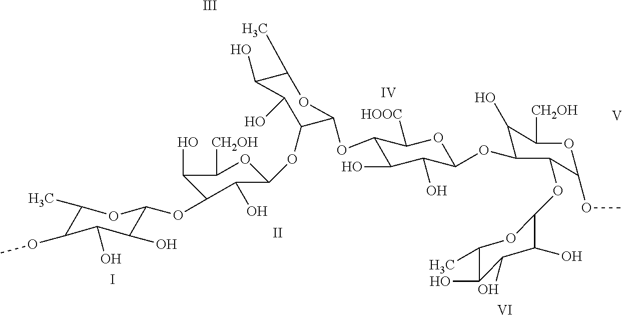

Preferably, the polysaccharide according to the invention is used in an aqueous solution at 2.5% by weight of active material, relative to the total weight of the solution. Such a polysaccharide is marketed particularly under the name Rhamnosoft® HP 1.5P by Solabia.

Preferably, the polysaccharide may be present in the composition according to the invention with a dry matter content ranging from 0.01% to 1% by weight relative to the total weight of the composition, preferably from 0.05% to 0.5% by weight, and more preferably from 0.1% to 0.3% by weight.

Ester of Fatty Acid and of Polyglycerol Comprising 5 to 9 Glycerol Patterns

The composition according to the invention also comprises at least 1% by weight relative to the total weight of the composition, at least one ester of fatty acid and of polyglycerol comprising from 5 to 9 glycerol patterns.

The ester of fatty acid and of polyglycerol is formed from at least one acid comprising an alkyl or alkenyl chain containing from 12 to 20 carbon atoms and from 5 to 9 glycerol patterns, preferably from 5 to 6 glycerol patterns.

According to one embodiment, the polyglycerol ester according to the invention results from esterification of at least one saturated or unsaturated fatty acid and a polyglycerol.

Preferably, the ester of fatty acid and of polyglycerol comprising from 5 to 9 glycerol patterns is a mono- or diester, and preferably a mono-ester.

The term “polyglycerol” designates glyceryl polymers that are linear chains of 5 to 9, and preferably 5 to 6 glyceryl units.

The esters considered most particularly in this invention are esters resulting from the esterification of polyglycerol and of C12-C20, preferably C12 to C18 and more preferably C12, carboxylic acids, such as lauric, oleic, stearic, isostearic or myristic acids.

The carboxylic acid may be linear or branched, saturated or unsaturated.

Preferably, it is a linear monocarboxylic acid.

In general, they are derived from esterification of at least one hydroxyl function of a polyglycerol by a C12-C20, preferably C12 to C18, and more particularly C6 to C18, and particularly C10 to C12, carboxylic acid.

According to one particular embodiment, esters suitable for this invention can be derived from esterification of a polyglycerol by one or several identical or different carboxylic acids. It may be a hydroxylated mono-ester, a hydroxylated di-ester, a hydroxylated tri-ester, or a mixture thereof.

In one preferred embodiment of the invention, the ester of fatty acid and of polyglycerol is chosen from among polyglyceryl monolaurate comprising 5 to 6 glycerol patterns, polyglyceryl monooleate comprising from 5 to 6 glycerol patterns, polyglyceryl mono(iso)stearate comprising 5 to 6 glycerol patterns, polyglyceryl dioleate comprising 5 to 6 glycerol patterns, polyglyceryl monomyristate comprising 5 to 6 glycerol patterns, and mixtures thereof.

In another preferred embodiment of the invention, the ester of fatty acid and of polyglycerol has an HLB (Hydrophilic Lipophilic Balance) value equal to between 10 and 13.

Advantageously, the composition according to the invention comprises an ester of fatty acid and of polyglycerol that is a polyglyceryl monolaurate with 5 to 6 glycerol patterns, i.e. polyglyceryl-5 laurate or polyglyceryl-6 laurate.

A commercial product predominantly based on polyglyceryl-5 laurate or PG-5 laurate is available under the tradename SUNSOFT A-121 E-C® by Taiyo Kagaku.

A commercial product predominantly based on polyglyceryl-6 laurate or PG-6 laurate is available under the tradename DERMOFEEL G 6 L by Dr Straetmans.

The ester of fatty acid and of polyglycerol comprising 5 to 9 glycerol patterns may be present in the composition according to the invention in a content ranging from 1% to 10% by weight relative to the total weight of the composition, preferably from 3% to 7% by weight, and more preferably from 4% to 6% by weight.

Polyol

The composition according to the invention also comprises at least one polyol.

For the purposes of the invention, polyol means a hydrocarbon chain comprising at least 2 carbon atoms, preferably from 2 to 50 carbon atoms, preferably from 4 to 20 carbon atoms, preferably from 2 to 10 carbon atoms, and more preferably from 2 to 6 carbon atoms and comprising at least two hydroxy groups. Polyols used in this invention can have an average molecular mass by weight equal to less than or equal to 1,000, preferably between 90 and 500.

The polyol may be a natural or synthetic polyol. The polyol can have a linear, branched or cyclic molecular structure.

The polyol can be chosen from among glycerin and its derivatives, and glycols and their derivatives. The polyol can be chosen from the group composed of glycerin, diglycerin, polyglycerin, diethylene glycol, propylene glycol, dipropylene glycol, butylene glycol, pentylene glycol, hexylene glycol, 1,3-propanediol, 1,5-pentanediol, octane 1,2-diol, polyethyleneglycols, particularly having 5 to 50 ethylene oxide groups, and sugars such as sorbitol, and mixtures of them.

More particularly, the polyol is glycerin.

Said polyol(s) can be present in a quantity ranging from 2% to 30% by weight, relative to the total weight of the composition, preferably ranging from 3% to 25% by weight, and preferably ranging from 5% to 20% by weight.

Preferably, the composition according to the invention is an emulsion. Preferably, the composition according to the invention comprises an aqueous phase and an oily phase, said aqueous and oily phases being as defined above. Preferably, the composition according to the invention is an oil-in-water emulsion.

When the composition according to the invention comprises at least one surfactant as described below, it preferably has the aspect of a cream, particularly a white cream.

When the composition according to the invention does not comprise a surfactant as described below, it corresponds to a micro-emulsion.

Surfactants

The composition according to the invention preferably comprises at least one ester of fatty acid and of polyethylene glycol, as surfactant. Preferably, it also comprises an additional surfactant chosen from among C₁₆-C₂₂ fatty acid and sorbitan esters and C₁₆-C₂₂ fatty acid and glyceryl esters.

The ester of fatty acid and of polyethylene glycol present in the composition according to the invention is preferably a C₁₆-C₂₂ fatty acid ester comprising 8 to 100 ethylene oxide units. The fatty chain of esters can be chosen particularly among the stearyl, behenyl, arachidyl, palmityl, cetyl patterns and mixtures thereof, such as cetearyl, and preferably a stearyl chain.

The number of ethylene oxide units can vary from 8 to 100, preferably from 10 to 80, and even better from 10 to 50. According to one particular embodiment of the invention, this number can vary from 20 to 40.

As an example of a fatty acid and polyethylene glycol ester, mention may be made of stearic acid esters comprising 20, 30, 40, 50 or 100 units of ethylene oxide, such as products marketed under the tradename Myrj 49 P (polyethylene glycol stearate 20 OE; CTFA name: PEG-20 stearate), Myrj 51, Myrj 52 P (polyethyleneglycol stearate 40 OE; CTFA name: PEG-40 stearate), Myrj 53 or Myrj 59 P by CRODA.

The ester of fatty acid and of polyethylene glycol may be present in the composition according to the invention in a content ranging from 0.1% to 10% by weight relative to the total weight of the composition, preferably from 0.1% to 5% by weight, and more preferably from 0.25% to 1.5% by weight.

Preferably, the composition according to the invention also comprises an additional emulsifying surfactant chosen from among C₁₆-C₂₂ fatty acid and sorbitan esters and C₁₆-C₂₂ fatty acid and glyceryl esters.

According to a first embodiment of the invention, the composition comprises a C₁₆-C₂₂ fatty acid and sorbitan ester.

The C₁₆-C₂₂ fatty acid and sorbitan esters are formed by esterification of at least one fatty acid comprising at least one saturated or unsaturated linear alkyl chain with 16 to 22 carbon chains, with sorbitol. In particularly, these esters can be chosen from among stearates, behenates, arachidates, palmitates, oleates of sorbitan, and mixtures thereof. Sorbitan stearates and palmitates will be used in preference, and more preferably sorbitan stearates.

The C₁₆-C₂₂ fatty acid and sorbitan ester present in the composition according to the invention is advantageously solid at a temperature of less than or equal to 45° C.

As example of a sorbitan ester that can be used in the composition according to the invention, mention can be made of sorbitan monostearate (CTFA name: Sorbitan stearate) sold by Croda under the tradename Span 60, sorbitan tristearate sold by Croda under the trade name Span 65 V, sorbitan monopalmitate (CTFA name: Sorbitan palmitate) sold by Croda under the tradename Span 40, sorbitan monoleate sold by Croda under the name Span 80 V, sorbitan trioleate sold by Uniquema under the tradename Span 85 V, preferably the sorbitan ester used is sorbitan tristearate.

The C₁₆-C₂₂ fatty acid and sorbitan ester can be present in the composition according to the invention in a content ranging from 0.01% to 10% by weight relative to the total weight of the composition, preferably from 0.01% to 5% by weight, and more preferably from 0.25% to 1.5% by weight.

The glyceryl and fatty acid ester can be obtained particularly using an acid comprising a saturated linear alkyl chain, with 16 to 22 carbon atoms. As a glyceryl and fatty acid ester, particular mention may be made of glyceryl stearate (glyceryl mono-, di- and/or tri-stearate) (CTFA name: Glyceryl stearate), glyceryl ricinoleate, and mixtures thereof. Preferably the glyceryl and fatty acid ester used is chosen from among glyceryl stearates.

The glyceryl and fatty acid ester can be present in a quantity ranging from 0.1 to 10% by weight, relative to the total weight of the composition, preferably ranging from 0.1 to 5% by weight, and preferably ranging from 0.5% to 3% by weight.

In particular, the composition according to the invention may comprise a mixture of glyceryl stearate and polyethylene glycol 1000E monostearate, and in particular that comprising a 50/50 mixture marketed under the tradename Arlacel 165 by Croda.

Aqueous Phase

Preferably, in addition to polyol, the composition according to the invention comprises a physiologically acceptable aqueous medium. “Physiologically acceptable” means a medium compatible with keratin materials.

The composition according to the invention preferably comprises an aqueous medium comprising water and possibly an organic solvent soluble in water, at 25° C., chosen for example among linear or branched C2-C4 alkanols such as ethanol and isopropanol, propanol, butanol; and mixtures thereof.

The composition generally comprises from 10% to 95% by weight of water with respect to the total weight of the composition and preferably from 40 to 80%.

The quantity of organic solvents can range for example from 0% to 30% by weight, preferably from 0.5% to 25% by weight, better from 5% to 20% by weight, even better from 10% to 22% by weight relative to the total weight of the composition.

Hydrophilic Active Agent(s)

The composition according to the invention may comprise an aqueous at least one hydrophilic active agent. By “hydrophilic active agent” it is meant an active agent which is hydrosoluble or hydrodispersible, and which is capable of forming hydrogen bonds.

Examples of hydrophilic active agents that may be mentioned include moisturizing agents; depigmenting agents, desquamating agents, anti-aging agents, mattifying agents; healing agents; antibacterial agents; and their mixtures.

Preferably the hydrophilic active agent is chosen from the C-glycoside derivatives of general formula (F) below:

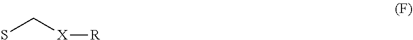

in which:

R denotes an unsubstituted linear C1-C4 alkyl radical, especially C1-C2, in particular methyl;

S represents a monosaccharide chosen from D-glucose, D-xylose, N-acetyl-D-glucosamine or L-fucose, and in particular D-xylose;
X represents a group chosen from —CO—, —CH(OH)—, —CH(NH2)-, and preferentially a —CH(OH)— group;
as well as their cosmetically acceptable salts, their solvates such as hydrates and their optical isomers.

By way of non-limiting illustration of the C-glycoside of formula (F) that is more particularly suitable for the invention, the following compounds may be mentioned:

C-beta-D-xylopyranoside-n-propan-2-one;
C-alpha-D-xylopyranoside-n-propan-2-one;
C-beta-D-xylopyranoside-2-hydroxy-propane;
C-alpha-D-xylopyranoside-2-hydroxy-propane;
1-(C-beta-D-glucopyranosyl)-2-hydroxy-propane;
1-(C-alpha-D-glucopyranosyl)-2-hydroxy-propane;
1-(C-beta-D-glucopyranosyl)-2-amino-propane;
1-(C-alpha-D-glucopyranosyl)-2-amino-propane;
3′-(acetamido-C-beta-D-glucopyranosyl)-propan-2′-one;
3′-(acetamido-C-alpha-D-glucopyranosyl)-propan-2′-one;
1-(acetamido-C-beta-D-glucopyranosyl)-2-hydroxy-propane;
1-(acetamido-C-beta-D-glucopyranosyl)-2-amino-propane;
as well as their cosmetically acceptable salts, their solvates such as hydrates and their optical isomers.

Preferably, C-beta-D-xylopyranoside-2-hydroxy-propane or C-alpha-D-xylopyranoside-2-hydroxy-propane, and more preferably C-beta-D-xylopyranoside-2-hydroxy-propane, are used. Preferably, a C-glycoside of formula (F) that is suitable for the invention may advantageously be C-beta-D-xylopyranoside-2-hydroxy-propane, whose INCI name is HYDROXYPROPYL TETRAHYDROPYRANTRIOL, sold especially under the name MEXORYL SBB® or MEXORYL SCN® by CHIMEX. The salts of C-glycosides of formula (F) suitable for the invention may comprise conventional physiologically acceptable salts of these compounds such as those formed from organic or inorganic acids. By way of example, mention may be made of mineral acid salts, such as sulfuric acid, hydrochloric acid, hydrobromic acid, hydroiodic acid, phosphoric acid and boric acid. Mention may also be made of organic acid salts, which may comprise one or more carboxylic acid, sulphonic acid or phosphonic acid groups. It may be linear, branched or cyclic aliphatic acids or aromatic acids. These acids may furthermore comprise one or more heteroatoms chosen from O and N, for example in the form of hydroxyl groups. These include propionic acid, acetic acid, terephthalic acid, citric acid or tartaric acid. Acceptable solvates for the compounds described above include conventional solvates such as those formed in the last step of preparing said compounds due to the presence of solvents. By way of example, mention may be made of solvates due to the presence of water or of linear or branched alcohols, such as ethanol or isopropanol. C-glycosides (I) are known from WO 02/051828.

According to one embodiment, the composition according to the invention comprises a C-glycoside in an amount of between 0.05% and 10% by weight of active ingredient (C-glycoside) relative to the total weight of the composition, in particular between 0.5% and 5% by weight of active material relative to the total weight of the composition, more particularly between 1% and 4% by weight of active material relative to the total weight of the composition.

Oily Phase

The composition according to the invention preferably also comprises at least one oily phase. When the composition used according to the invention comprises an oily phase, this oily phase preferably contains at least one oil, particularly a cosmetic oil. It may further contain other fats.

By way of oils suitable for use in the composition according to the invention, mention may be made for example of:
hydrocarbon oils of animal origin, such as perhydrosqualene;
hydrocarbon oils of plant origin, such as liquid fatty acid triglycerides having 4 to 10 carbon atoms such as heptanoic or octanoic acid triglycerides or, for example, sunflower, corn, soybean, pumpkin, grape seed, sesame, hazelnut, apricot, macadamia, arara, sunflower, castor, avocado oils, caprylic/capric acid triglycerides such as those sold by Stearineries Dubois or those sold under the trade names Miglyol “810”, “812” and “818” by Dynamit Nobel, jojoba oil, shea butter oil;
esters and synthetic esters, in particular fatty acids, such as oils having formulas R1COOR2 and R1OR2 wherein R1 is the remainder of a fatty acid comprising from 8 to 29 carbon atoms, and R2 is a hydrocarbon chain, branched or not, containing from 3 to 30 carbon atoms, such as for example Purcellin oil, isononyl isononanoate, isopropyl myristate, ethyl-2-hexyl palmitate, octyl-2-dodecyl stearate, octyl-2-dodecyl erucate, isostearyl isostearate; hydroxylated esters such as isostearyl lactate, octylhydroxystearate, octyldodecyl hydroxystearate, diisostearyl-malate, triisocetyl citrate; heptanoates, octanoates, decanoates of fatty alcohols; polyol esters, such as propylene glycol dioctanoate, neopentyl glycol diheptanoate and diethylene glycol diisononanoate; and pentaerythritol esters such as pentaerythrityl tetraisostearate;
linear or branched hydrocarbons, with inorganic or synthetic origin, such as volatile or non-volatile paraffin oils and derivatives thereof, hydrocarbon oils with branched chain containing 10 to 20 carbon atoms such as isohexadecane, isododecane, isoparaffins and mixtures thereof, vaseline, polydecenes, hydrogenated polyisobutene such as Parleam® oil;
fatty alcohols having 8 to 26 carbon atoms, such as cetyl alcohol, stearyl alcohol, the mixture of cetyl alcohol and stearyl alcohol (cetylstearyl alcohol), octyldodecanol, 2-butyloctanol, 2-hexyldecanol, 2-undecylpentadecanol, oleic acid or linoleic acid;
partially hydrocarbon and/or silicone fluorinated oils such as those described in the document JP-A-2-295912;
silicone oils such as polymethylsiloxanes (PDMS), optionally volatile with a linear or cyclic silicone chain, liquid or pasty at ambient temperature, particularly cyclopolydimethylsiloxanes (cyclomethicones) such as cyclohexasiloxane; polydimethylsiloxanes comprising alkyl, alkoxy or phenyl pendant or silicon chain-end groups, groups having 2 to 24 carbon atoms; phenyl silicones such as phenyltrimethicones, phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes; or
mixtures thereof.

The quantity of oil phase may range for example 0.1% to 30%, and for example from 10% to 20% by weight with respect to the total weight of the composition.

Preparation and Properties

The cosmetic composition according to this invention can be prepared by mixing the above essential and optional components using a conventional method. Preferably, the cosmetic composition is prepared by a method using low energy.

As described above, when the composition according to the invention does not comprise a surfactant as described in the corresponding part of this application, it corresponds to a micro-emulsion.

The “micro-emulsion” can be defined in two ways, in other words in a broad sense and in a more restricted sense. Specifically, in one case (“micro-emulsion in the restricted sense”), the micro-emulsion designates a single thermodynamically stable isotropic liquid phase containing a ternary system with three components comprising an oily component, an aqueous component and a surfactant, and in the other case (“micro-emulsion in the broad sense”), among typical thermodynamically unstable emulsion systems, the micro-emulsion also comprises emulsions with transparent or translucent appearance due to the smaller size of their particles (Satoshi Tomomasa, et al., Oil Chemistry, vol. 37, No. 11 (1988), p. 48-53). In this context, “micro-emulsion” designates a “micro-emulsion in the restricted sense”, in other words a single thermodynamically stable isotropic liquid phase.

Micro-emulsion designates a state of an O/W (oil-in-water) type micro-emulsion in which the oil is solubilized by micella, a W/E (water-in-oil) type micro-emulsion in which water is solubilized by inverse micella, or a bicontinuous micro-emulsion in which the number of associations of surfactant molecules is made infinite so that the aqueous phase and the oily phase both have a continuous structure.

The micro-emulsion may have a dispersed phase with a mean diameter by number equal to 300 nm or less, preferably 200 nm or less and more preferably 100 nm or less, as measured by laser granulometry.

We will now give concrete examples illustrating the invention, but that are in no way restrictive.

In the examples, the temperature is ambient temperature (20° C.) expressed in degrees Celsius unless mentioned otherwise, and the pressure is atmospheric pressure, unless mentioned otherwise.

In the examples, quantities of the ingredients of the compositions are given as a by weight relative to the total weight of the composition.

### EXAMPLE 1: PREPARATION OF A COMPOSITION ACCORDING TO THE INVENTION (F5) AND COMPARATIVE COMPOSITIONS (F1 TO F4)

The following compositions F1 to F5 were prepared with the ingredients mentioned in the following table, using the protocol described below:

The ingredients of phase B are mixed and heated to 80° C.;
The ingredients of phase A are mixed and heated to 80° C.;
Under Rayneri stirring, phase B is poured slowly into phase A;
Phase C is added at about 50° C.

TABLE 1

| | | | | | | F5 |
|---|---|---|---|---|---|---|
| Ingredients | Phase | F1* | F2* | F3* | F4* | (invention) |
| Water | A | Qs 100 | Qs 100 | Qs 100 | Qs 100 | Qs 100 |
| Preservative | A | Qs | Qs | Qs | Qs | Qs |
| Biosaccharide gum-2 | A | | 5 | | | 5 |
| (Rhamnosoft HP 1.5P by | | | | | | |
| Solabia) | | | | | | |
| (2.5% active material) | | | | | | |
| Glycerol | A | | | 10 | | 10 |
| POLYGLYCERYL-5 | B | | | | 5 | 5 |
| LAURATE | | | | | | |
| (SUNSOFT A-121E-C ® | | | | | | |
| by Taiyo Kagaku) | | | | | | |
| GLYCERYL STEARATE | B | 2.5 | 2.5 | 2.5 | 2.5 | 2.5 |
| (and) PEG-100 | | | | | | |
| STEARATE | | | | | | |
| (Arlacel 165 by Croda) | | | | | | |
| PEG-40 STEARATE | B | 2.5 | 2.5 | 2.5 | 2.5 | 2.5 |
| (Myrj 52 P by Croda) | | | | | | |
| Cetyl alcohol | B | 1 | 1 | 1 | 1 | 1 |
| Stearyl alcohol | B | 1 | 1 | 1 | 1 | 1 |
| Hydrogenated | B | 6 | 6 | 6 | 6 | 6 |
| polyisobutene | | | | | | |
| Cyclohexasiloxane | B | 4 | 4 | 4 | 4 | 4 |
| Sodium hydroxide | C | 0.07 | 0.07 | 0.07 | 0.07 | 0.07 |
| CARBOMER | C | 0.25 | 0.25 | 0.25 | 0.25 | 0.25 |
| (CARBOPOL 980 | | | | | | |
| POLYMER by Lubrizol | | | | | | |
| 98% CARBOXYVINYLIC | | | | | | |
| POLYMER) | | | | | | |

Compositions F1 to F4 are comparative (marked by a star).
Composition F5 is according to the invention.

### EXAMPLE 2: IN VITRO MEASUREMENT OF THE MECHANICAL EFFECT OF A COMPOSITION ACCORDING TO THE INVENTION (F5) AND COMPARATIVE COMPOSITIONS (F1 TO F4)

The compositions F1 to F5 in example 1 are tested for their mechanical properties by elasticimetry, as follows:

A DMA (Dynamic Mechanical Analysis) apparatus sold under reference ElectraForce® 3100 by Bose was used.

This technique was used to study viscoelastic properties of the stratum corneum. The material was loaded sinusoidally and its deformation was measured. The storage modulus (E′) of the stratum corneum can then be determined, that characterizes it mechanically.

This magnitude is directly related to the elastic properties of the stratum corneum.

The impact of formulas F1 to F5 on this storage modulus were studied.

### Preparation of Samples and Protocol:

The area of the stratum corneum sample to be tested is 2 cm² (1 cm×2 cm). The samples were previously conditioned at 75% relative humidity for at least 12 hours, and the measurement was also made at 75% relative humidity.

10 μl/cm² of each formula to be tested was deposited on the stratum corneum sample. Each formula was spread on the stratum corneum so as to cover the entire surface.

The dynamic load amplitude was adjusted to 40 μm, which correspond to a deformation in the elastic range of the stratum corneum (0.2% deformation). Each sample was loaded at a frequency of 1 Hz, along its longest length. Stratum corneum from at least two different donors was used.

For each formula evaluated, the variation of the elastic modulus measured with each treated sample was calculated, during the first two hours of treatment (E2 h), compared with that of an untreated sample at t=0 (E0):

ΔE=(E2h−E0)/E0

The following results were obtained:

TABLE 2

| | | | | | F5 |
|---|---|---|---|---|---|
| Ingredients | F1* | F2* | F3* | F4* | (invention) |
| average loss % | 47.17 | 53.53 | 49.32 | 29.12 | 78.12 |
| Standard | 11.80 | 20.86 | 13.96 | 13.42 | 7.53 |
| deviation | | | | | |
| | | | | T test | 0.001 |
| | | | | versus | (p < 0.05) |
| | | | | formula F1 | |
| | | | | T test | 0.026 |
| | | | | versus | (p < 0.05) |
| | | | | formula F2 | |
| | | | | T test | 0.011 |
| | | | | versus | (p < 0.05) |
| | | | | formula F3 | |
| | | | | T test | 0.000 |
| | | | | versus | (p < 0.05) |
| | | | | formula F4 | |

In conclusion, it is found that the mechanical effect is provided by the association of PG-5 laurate, glycerin and polysaccharide.

### EXAMPLE 3: IN VIVO COMPARISON OF THE COMPOSITION ACCORDING TO THE INVENTION (F5) WITH COMPARATIVE COMPOSITIONS COMPRISING OTHER POLYSACCHARIDES (F6 TO F7)

Composition F5 in example 1 is compared with the following comparative formulations F6 to F7 (see table below; the comparative formulas are marked by a star). The formula preparation protocol is identical to that described in example 1.

In particular, the comparative formula F6 comprises Glycofilm 1.5P that is a polysaccharide rich in fucose, glucose and glucorinic acid.

The comparative formula F7 comprises Fucogel 1.5P that is a polysaccharide rich in fucose (i.e. 20%).

These formulas are prepared then analyzed in vivo with the Torquemeter as follows:

The Torquemeter® is a non-invasive device. The measurement head of the DTM is composed of a 20 mm diameter mobile central disk and a fixed circular plate. This device is placed on the skin through a fixed concentric double-sided adhesive tape. The angle of rotation of the central disk is measured by an angular sensor with a very high resolution. During application of the measurement head, the central disk pivots. A torsion load equal to an angle Ue is then applied to the skin area between the mobile central disk and the fixed peripheral ring (fast deformation). The rotation angle then continues to rise at a lower speed by an angle Uv.

After removing the torsion torque, the skin returns to its initial state in two steps, fast (deformation Ur) and slow back to the origin.

The precise measurement zones are identified using a circular-shaped mask. The measured parameters are (Ue, Uv, Ur).

The results are also given in the following table.

TABLE 3

| | | | | F5 |
|---|---|---|---|---|
| Phase | Ingredients | F6* | F7* | (invention) |
| A | Water | Qs 100 | Qs 100 | Qs 100 |
| A | Preservative | Qs | Qs | Qs |
| A | Biosaccharide gum-2 | | | 5 |
| | (Rhamnosoft HP 1.5P by | | | (0.125**) |
| | Solabia) | | | |
| | (2.5% active material) | | | |
| A | BIOSACCHARIDE GUM-4 | 12.5 | | |
| | (1.2% active material) | (0.15**) | | |
| | (GLYCOFILM 1.5P by | | | |
| | Solabia) | | | |
| A | BIOSACCHARIDE GUM-1 | | 11.36 | |
| | (1.1% active material) | | (0.125**) | |
| | (FUCOGEL 1.5P by | | | |
| | Solabia) | | | |
| A | Glycerin | 10 | 10 | 10 |
| B | POLYGLYCERYL-5 | 5 | 5 | 5 |
| | LAURATE | | | |
| | (SUNSOFT A-121E-C ® by | | | |
| | Taiyo Kagaku) | | | |
| B | GLYCERYL STEARATE | 2.5 | 2.5 | 2.5 |
| | (and) PEG-100 | | | |
| | STEARATE | | | |
| | (Arlacel 165 by Croda) | | | |
| B | PEG-40 STEARATE | 2.5 | 2.5 | 2.5 |
| | (Myrj 52 P by Croda) | | | |
| B | Cetyl alcohol | 1 | 1 | 1 |
| B | Stearyl alcohol | 1 | 1 | 1 |
| B | Hydrogenated | 6 | 6 | 6 |
| | polyisobutene | | | |
| B | CYCLOHEXASILOXANE | 4 | 4 | 4 |
| C | SODIUM HYDROXIDE | 0.07 | 0.07 | 0.07 |
| C | CARBOMER | 0.25 | 0.25 | 0.25 |
| | DTM (approximate values) | 0.5 | 0.45 | 0.95 |
| | 1h Delta Ur | | | |
| | DTM (approximate values) | 0.32 | 0.4 | 0.87 |
| | 1h Delta Uv | | | |
| | Ratio Ur/Uv | 1.56 | 1.13 | 1.09 |

**quantity of active material

The results show that only the polysaccharide comprising rhamnose according to the invention (biosaccharide gum-2), associated with polyglyceryl-5 laurate and glycerin, is significantly effective to make the stratum corneum more supple.

### EXAMPLE 4: IN VIVO TESTS WITH TWO COMPOSITIONS ACCORDING TO THE INVENTION (F5 AND F8) AND COMPARISON OF THE STABILITY OF COMPARATIVE FORMULAS (F9 TO F13)

1/Composition F5 in example 1 is tested as composition F8 according to the invention (see table below). The formula preparation protocol is identical to that described in example 1.

In particular, the formula according to the invention F8 comprises PG6-laurate instead of PG5-laurate.

These formulas are prepared then analyzed in vivo with the Torquemeter as described in example 3

The results are also given in the following table.

TABLE 4

| | | F5 | F8 |
|---|---|---|---|
| Phase | Ingredients | (invention) | (invention) |
| A | Water | Qs 100 | Qs 100 |
| A | Preservative | Qs | Qs |
| A | Biosaccharide gum-2 | 5 | 5 |
| | (Rhamnosoft HP 1.5P by Solabia) | | |
| | (2.5% active material) | | |
| A | Glycerol | 10 | 10 |
| B | POLYGLYCERYL-5 LAURATE | 5 | |
| | (SUNSOFT A-121E-C ® by | | |
| | Taiyo Kagaku) | | |
| B | POLYGLYCERYL-6 LAURATE | | 5 |
| | (Dermofeel G6L by Dr Straetmans) | | |
| B | GLYCERYL STEARATE | 2.5 | 2.5 |
| | (and) PEG-100 STEARATE | | |
| | (Arlacel 165 by Croda) | | |
| B | PEG-40 STEARATE | 2.5 | 2.5 |
| | (Myrj 52 P by Croda) | | |
| B | Cetyl alcohol | 1 | 1 |
| B | Stearyl alcohol | 1 | 1 |
| B | Hydrogenated polyisobutene | 6 | 6 |
| B | CYCLOHEXASILOXANE | 4 | 4 |
| C | CARBOMER | 0.25 | 0.25 |
| C | SODIUM HYDROXIDE | 0.07 | 0.07 |
| | DIM (approximate values) | 0.95 | 0.65 |
| | 1 Delta Ur | | |
| | DIM (approximate values) | 0.87 | 0.6 |
| | 1h Delta Uv | | |
| | Ratio Ur/Uv | 1.09 | 1.08 |

The results show that the DTM values obtained with formula F8 are also higher as for formula F5.

In conclusion, the increased suppleness effect obtained with PG5-laurate (F5) can also be obtained with PG6-laurate (F8).

2/Comparative formulas: the following comparative formulas F9 to 13 were prepared as described in example 1.

Their composition is given in Tables 5 and 6 below.

TABLE 5

| | F9 | F10 | F11 |
|---|---|---|---|
| Ingredient | (comparative) | (comparative) | (comparative) |
| Biosaccharide gum-2 | 5 | 5 | 5 |
| (Rhamnosoft HP 1.5P by | | | |
| Solabia) | | | |
| (2.5% active material) | | | |
| AMORPHOUS SILICA | 3 | 3 | 3 |
| MICROSPHERES (5 μm) | | | |
| SPHERICAL | 4.7 | 4.7 | 4.7 |
| CELLULOSE | | | |
| BALLS (SIZE 4-7 μM) | | | |
| Preservative | Qs | Qs | Qs |
| ISONONYL | 18.54 | 18.54 | 18.54 |
| ISONONANOATE | | | |
| DIPENTAERYTHRITYL | 2.85 | 2.85 | 2.85 |
| PENTAISONONANOATE | | | |
| SODIUM | 0.4 | 0.4 | 0.4 |
| ACRYLAMIDO-2- | | | |
| METHYL PROPANE | | | |
| SULFONATE/ | | | |
| HYDROXYETHYL- | | | |
| ACRYLATE | | | |
| COPOLYMER IN | | | |
| POWDER | | | |
| FORM (Sepinov | | | |
| EMT 100 by Seppic) | | | |
| AMPS/ETHOXYLATED | 0.7 | 0.7 | 0.7 |
| STEARYL | | | |
| METHACRYLATE | | | |
| (25 EO) COPOLYMER | | | |
| CROSS-LINKED BY | | | |
| TRIMETHYLOL- | | | |
| PROPANE | | | |
| TRIACRYLATE | | | |
| (TMPTA) | | | |
| (Aristoflex HMS | | | |
| by Clariant) | | | |
| Ethanol | 3 | 3 | 3 |
| WATER | Qs 100 | Qs 100 | Qs 100 |
| GLYCERIN | 10 | 10 | 10 |
| GLYCERYL | 1 | 1 | 1 |
| STEARATE (and) | | | |
| PEG-100 STEARATE | | | |
| (Arlacel 165 by Croda) | | | |
| PEG-40 STEARATE | 1 | 1 | 1 |
| (Myrj 52 P by Croda) | | | |
| PG-10 laurate | 5 | 2.5 | 1 |
| (DECAGLYCERYL | | | |
| MONOLAURATE) | | | |
| (DERMOFEEL G | | | |
| 10 L by Dr Straetmans) | | | |

For these formulas comprising 1%, 2.5% or 5% by weight of PG-10 laurate (F11, F10 and

F9 respectively), a reduction in the stability and slip on application are observed. Thus, PG-10 laurate does not confer the same effects as the fatty acid and polyglycerol ester comprising 5 to 9 glycerol patterns according to the invention.

TABLE 6

| | F12 | F13 |
|---|---|---|
| | (compar- | (compar- |
| Ingredient | ative) | ative) |
| Biosaccharide gum-2 | 5 | 5 |
| (Rhamnosoft HP 1.5P by Solabia) | | |
| (2.5% active material) | | |
| AMORPHOUS SILICA | 3 | 3 |
| MICROSPHERES (5 μm) | | |
| SPHERICAL CELLULOSE BALLS | 4.7 | 4.7 |
| (SIZE 4-7 μM) | | |
| Preservative | Qs | Qs |
| ISONONYL ISONONANOATE | 18.54 | 18.54 |
| DIPENTAERYTHRITYL | 2.85 | 2.85 |
| PENTAISONONANOATE | | |
| SODIUM ACRYLAMIDO-2-METHYL | 0.4 | 0.4 |
| PROPANE SULFONATE/ | | |
| HYDROXYETHYLACRYLATE | | |
| COPOLYMER IN POWDER FORM | | |
| (Sepinov EMT 100 by Seppic) | | |
| AMPS/ETHOXYLATED STEARYL | 0.7 | 0.7 |
| METHACRYLATE (25 EO) | | |
| COPOLYMER CROSS-LINKED BY | | |
| TRIMETHYLOLPROPANE | | |
| TRIACRYLATE (TMPTA) | | |
| (Aristoflex HMS by Clariant) | | |
| Ethanol | 3 | 3 |
| WATER | Qs 100 | Qs 100 |
| GLYCERIN | 10 | 10 |
| GLYCERYL STEARATE (and) PEG-100 | 1 | 1 |
| STEARATE (Arlacel 165 by Croda) | | |
| PEG-40 STEARATE | 1 | 1 |
| (Myrj 52 P by Croda) | | |
| POLYGLYCERYL-4 LAURATE | 5 | 2.5 |
| (TEGO CARE PL 4 by Evonik) | | |

The formulas cannot be produced using PG-4 laurate: the incorporation of 2.5% or 5% by weight of PG-4 laurate, as in comparative formulas F12 and F13, leads to sedimentation and salting out: the formulas cannot be made.

Thus, PG-4 laurate does not confer the same effects as the ester of fatty acid and of polyglycerol comprising 5 to 9 glycerol patterns according to the invention.

### EXAMPLE 5: FORMULA ACCORDING TO THE INVENTION

The following formula according to the invention comprising a hydrophilic active agent is prepared.

TABLE 7

| | Formula |
|---|---|
| Ingredient | (invention) |
| Biosaccharide gum-2 | 7.5 |
| (Rhamnosoft HP 1.5P by Solabia) | |
| (2.5% active material) | |
| AMORPHOUS SILICA | 3 |
| MICROSPHERES (5 μm) | |
| SPHERICAL CELLULOSE BALLS | 4.7 |
| (SIZE 4-7 μM) | |
| Preservative | Qs |
| ISONONYL ISONONANOATE | 18.54 |
| DIPENTAERYTHRITYL | 2.85 |
| PENTAISONONANOATE | |
| SODIUM ACRYLAMIDO-2-METHYL | 0.4 |
| PROPANE SULFONATE/ | |
| HYDROXYETHYLACRYLATE | |
| COPOLYMER IN POWDER FORM | |
| (Sepinov EMT 100 by Seppic) | |
| AMMONIUM ACRYLOYLDIMETHYL- | 0.7 |
| TAURATE/STEARETH-25 | |
| METHACRYLATE CROSSPOLYMER | |
| (Aristoflex HMS by Clariant) | |
| Ethanol | 3 |
| WATER | Qs 100 |
| GLYCERIN | 10 |
| GLYCERYL STEARATE (and) | 1 |
| PEG-100 STEARATE | |
| (Arlacel 165 by Croda) | |
| PEG-40 STEARATE | 1 |
| (Myrj 52 P by Croda) | |
| POLYGLYCERYL-5 LAURATE | 5 |
| (SUNSOFT A-121E-C ® by Taiyo | |
| Kagaku) | |
| Sodium hyaluronate | 0.5 |
| Hydroxypropyl tetrahydropyrantriol | 8.7 |
| (MEXORYL SCN ® of CHIMEX | (=3.045% active |
| (35% active material) | material) |

1. A composition comprising, in a physiologically acceptable medium:
at least one polysaccharide comprising rhamnose,
at least one polyol, and
at least 1% by weight relative to the total weight of composition, at least one ester of fatty acid and of polyglycerol comprising from 5 to 9 glycerol patterns.

2. The composition according to claim 1, wherein the polysaccharide has a rhamnose content varying from 10% to 100% by weight relative to the total weight of polysaccharide.

3. The composition according to claim 1, wherein the repetitive elements that form the polysaccharide comprise at least components with general formula I:
(Rh;Rh;Rh*;U;O)ₙ (I)
wherein Rh is a rhamnose molecule, Rh* is a rhamnose molecule fixed in a branched manner, O is a molecule of a hexosidic or pentosidic sugar, U is a molecule of uronic acid and n is between 1 and 100.

4. The composition according to claim 1, wherein the repetitive elements forming the polysaccharide are composed of the sequence with general formula II: 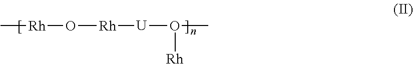
wherein Rh is a rhamnose molecule, O is a hexosidic or pentosidic sugar molecule, U is a uronic acid molecule and the rhamnose branch onto the ose O consists of an osidic bond (1→2) or (1→3).

5. The composition according to claim 1, wherein the repetitive elements forming the polysaccharide are composed of the sequence with general formula III: 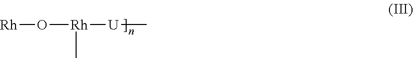
wherein Rh is a rhamnose molecule, O is a hexosidic or pentosidic sugar molecule, U is a uronic acid molecule and rhamnose is branched onto the rhamnose by an osidic bond (1→3).

6. The composition according to claim 1, wherein the polysaccharide is a polymer with a branched structure, with molecular weight of the order of 50,000 daltons, and with a saccharidic sequence comprising three rhamnose molecules (I, III, VI), two galactose molecules (II, V) and one glucuronic acid molecule (IV), said sequence having the following detailed formula:

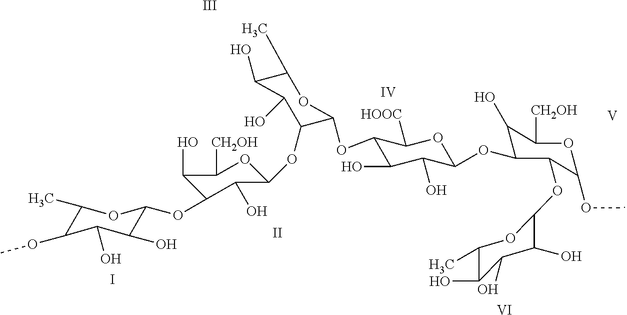

7. The composition according to claim 1, wherein the ester of fatty acid and of glycerol comprising 5 to 9 glycerol patterns is polyglyceryl-5 laurate or polyglyceryl-6 laurate.

8. The composition according to claim 1, wherein the ester of fatty acid and of polyglycerol is present in the composition according to the invention in a content ranging from 1% to 10% by weight relative to the total weight of the composition.

9. The composition according to claim 1, wherein the polyol is chosen from among glycerin and its derivatives, and glycols and their derivatives; and their mixtures.

10. The composition according to claim 1, wherein the polyol is present in a content ranging from 2% to 30% by weight, relative to the total weight of the composition.

11. The composition according to claim 1, wherein it comprises at least one surfactant that is a fatty acid and polyethylene glycol ester.

12. The composition according to claim 1, wherein it comprises an aqueous phase and/or an oily phase.

13. The composition according to claim 1, which comprises at least one hydrophilic active agent.

14. A cosmetic method for cleansing keratin materials comprising the application of a composition according to claim 1 onto said keratin materials.

15. A cosmetic method for making the skin more supple, particularly the stratum corneum, comprising the application of a composition according to claim 1 onto the skin.

16. A composition comprising, in a physiologically acceptable medium:
at least one polysaccharide comprising rhamnose, and
at least at least one hydrophilic active agent.

17. The composition according to claim 2, wherein the repetitive elements that form the polysaccharide comprise at least components with general formula I:
(Rh;Rh;Rh*;U;O)ₙ (I)
wherein Rh is a rhamnose molecule, Rh* is a rhamnose molecule fixed in a branched manner, O is a molecule of a hexosidic or pentosidic sugar, U is a molecule of uronic acid and n is between 1 and 100.

18. The composition according to claim 2, wherein the repetitive elements forming the polysaccharide are composed of the sequence with general formula II: 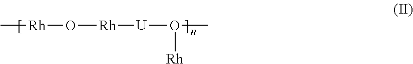
wherein Rh is a rhamnose molecule, O is a hexosidic or pentosidic sugar molecule, U is a uronic acid molecule and the rhamnose branch onto the ose O consists of an osidic bond (1→2) or (1→3).

19. The composition according to claim 3, wherein the repetitive elements forming the polysaccharide are composed of the sequence with general formula II: 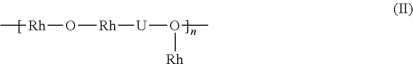
wherein Rh is a rhamnose molecule, O is a hexosidic or pentosidic sugar molecule, U is a uronic acid molecule and the rhamnose branch onto the ose O consists of an osidic bond (1→2) or (1→3).

20. The composition according to claim 2, wherein the repetitive elements forming the polysaccharide are composed of the sequence with general formula III: 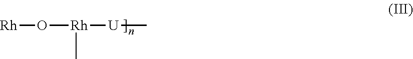
wherein Rh is a rhamnose molecule, O is a hexosidic or pentosidic sugar molecule, U is a uronic acid molecule and rhamnose is branched onto the rhamnose by an osidic bond (1→3).
